(19) United States
(12) Patent Application Publication
(10) Pub. No.: US 2001/0031950 A1
(43) Pub. Date: Oct. 18, 2001

(54) SURGICAL BLADE COATINGS

(75) Inventor: PHILLIP RYAN, MEMPHIS, TN (US)

Correspondence Address: JOEL R PETROW ESQ, SMITH & NEPHEW NORTH AMERICA, 1450 BROOKS ROAD, MEMPHIS, TN, 38116

(73) Assignee: Samantha Bell

(21) Appl. No.: 09/356,228

(22) Filed: Jul. 16, 1999

(51) Int. Cl. A61M 5/178; A61B 17/32

(52) U.S. Cl. 604/265; 606/180; 604/166.01

(57) ABSTRACT

A surgical blade includes an elongate outer tubular member, an elongate inner movably received within the outer member, and a polymer coating disposed on a portion of the outer surface of the inner member, the inner surface of the outer member, of both. The inner member has a distal cutter positionable adjacent a distal opening in the outer member. A method of cutting tissue, e.g., hard tissue such as bone, includes providing a surgical blade having an inner member and/or an outer member coated with a polymer, placing the surgical blade against the tissue, and driving the inner member to cut the tissue.

### BACKGROUND OF THE INVENTION

The present invention relates to surgical blades and, more particularly, to endoscopic surgical blades having elongate, inner and outer tubular members with distal ends cooperating to cut or resect bodily tissue.

Endoscopic surgical blades typically have an elongate outer tubular member terminating at a distal end having an opening in the side wall and/or the end wall to form a cutting port or window and an elongate inner tubular member coaxially disposed in the outer tubular member and having a distal end disposed adjacent the opening in the distal end of the outer tubular member. The distal end of the inner tubular member has a surface or edge for engaging tissue via the opening in the distal end of the outer tubular member and in many cases cooperates with the opening to shear or cut tissue. The inner tubular member is typically rotatable relative to the outer tubular member.

The distal end of the inner tubular member can have various configurations dependent upon the surgical procedure to be performed. The opening in the distal end of the outer tubular member is configured to cooperate with the particular configurations of the distal end of the inner tubular member. For example, the inner and outer tubular members can be configured to produce whisker cutting, arthroplasty burring or abrading, side cutting, meniscus cutting, trimming, full radius resection, end cutting and the like, and the various configurations are referred to herein generically as “cutting blades or edges.” Cut tissue is typically aspirated through the lumen of the inner tubular member.

The inner and outer members are typically formed from electropolished stainless steel. It is known to coat the surfaces of the members with a layer of silver, gold, tin-nickel alloy, or titanium nitride to act as a bearing surface between the outer and inner members.

### SUMMARY OF THE INVENTION

According to one aspect of the invention, a surgical blade includes an elongate outer tubular member and an elongate inner member movably received within the outer member and a polymer coating disposed on a portion of the outer surface of the inner member, the inner surface of the outer member, or both. The inner member has a distal cutter positionable adjacent a distal opening in the outer member.

The polymer coating reduces friction between the elongate outer tubular member and elongate inner member. Thus, the surgical blade, in operation, requires significantly less energy for moving the elongate outer tubular member and elongate inner member relative to each other. Furthermore, the need for additional lubricating oils (e.g., silicone oil,) often required with certain metallic coatings (e.g. silver or copper,) is eliminated. Eliminating the use of lubricating oils, eliminates any concern such oils being “washed” off of the surface of the blade and into the surgical site. The polymer coating also minimizes the introduction of fine metal particles into the surgical site, by eliminating the metal upon metal bearing surface and substituting it with a friction-reduced coating.

Embodiments of this aspect of the invention may include one or more of the following features.

The coating is a lubricious polymer coating, for example, selected from the group consisting of fluoropolymers, polyimide, polyamide, polyoxymethylene (acetal), polyorganosiloxane (silicone), hydrogels, and polyamide-imide. In the case that the lubricious polymer coating is a fluoropolymer, the coating includes polytetrafluoroethylene (PTFE.)

The coating is in a region of the distal opening of the outer tubular member, a region of the distal cutter of the inner member, or both. The coating is along substantially an entire length of the inner surface of the outer tubular member, the outer surface of the inner member, or both. Alternatively, the coating is provided along a limited portion of the length of the inner surface of the outer tubular member, the outer surface of the inner member, or both.

The inner member defines a lumen. The inner member is received within the outer tubular member for rotation therein. Alternatively, the inner member is received within the outer tubular member for axial, reciprocating motion therein.

The clearance between a distal region of the outer member and a distal region of the inner member prior to applying the coating is in a range of about 0.0001″ to 0.002″, preferably about 0.00075″ to 0.00175″. The thickness of the coating is in a range of about 0.0005″ to 0.0025″.

The outer and inner members are formed from stainless steel, e.g, soft stainless steel such as 300 series stainless steel.

According to another aspect of the invention, a method of cutting tissue includes providing a cutting blade having a portion of the outer surface of the inner member, the inner surface of the outer members, or both coated with a polymer coating, placing the cutting blade against the tissue, and driving the inner member to cut the tissue.

Embodiments of this aspect of the invention may include one or more of the following features.

The clearance between a distal region of the outer member and a distal region of the inner member prior to applying the coating is in a range of about 0.0001″ to 0.002″, preferably about 0.00075″ to 0.00175″. The coating has a thickness in a range of about 0.0005″ to 0.0025″. The method includes cutting or grinding hard tissue such as bone. The step of driving includes rotating the inner member within the outer member. The blade is sterilizable using an alcohol-based sterilant, such as ethylene oxide. The outer and inner members are formed from stainless steel, e.g., soft stainless steel.

According to another aspect of the invention, a method of making a surgical blade includes providing the outer tubular member and the inner member, and coating the inner surface of the outer tubular member, the outer surface of the inner tubular member, or both with a polymer.

Advantages of the invention may include one or more of the following: The polymer coating limits shedding. The coating provides good performance of the surgical blade particularly at high speeds, and also provides good performance of the surgical blade under high loads, e.g., when cutting or grinding bone. The coating can be deposited by conventional techniques in an economical manner, can be sterilized using conventional processes, such as gamma radiation and alcohol-based sterilants, without changing color, and is non-cytotoxic.

Other features and advantages of the invention will be apparent from the following detailed description.

### BRIEF DESCRIPTION OF THE DRAWINGS

FIG. 1 is a side elevation of a surgical cutting instrument according to the present invention.

FIG. 2 is a side elevation of an inner tubular member of the surgical cutting instrument of FIG. 1;

FIG. 3 is a detail view in section, in enlarged scale, taken along lines 3-3 of FIG. 2; and

FIG. 4 is a detail view, similar to FIG. 3, of another embodiment of the present invention.

### DETAILED DESCRIPTION

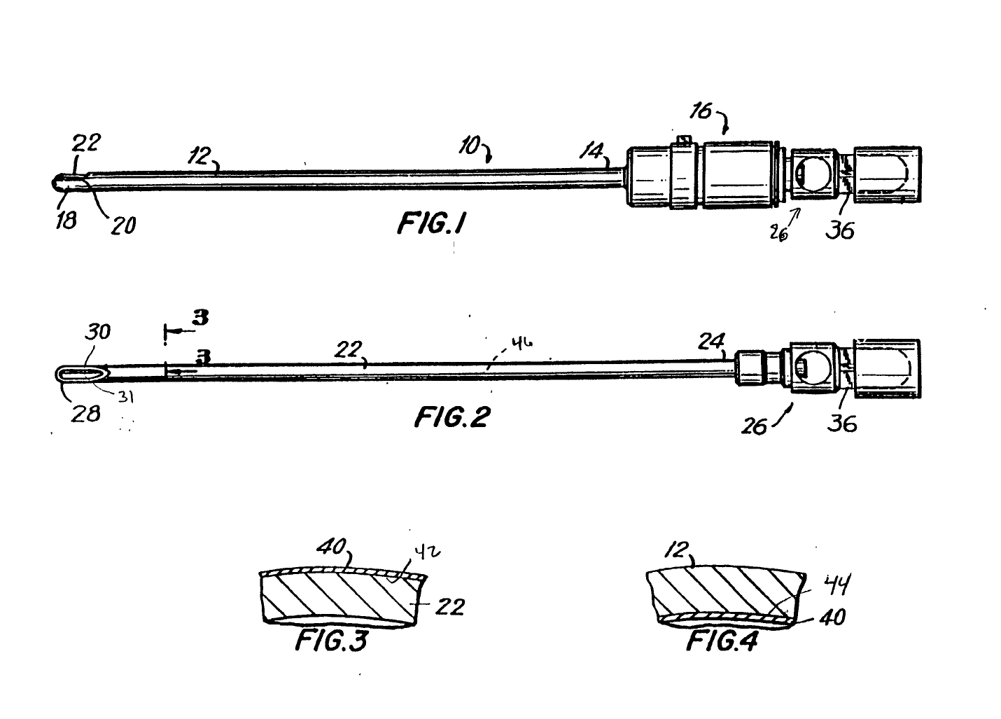

Referring to FIG. 1, a surgical blade 10 includes an elongate tubular outer member 12 having a proximal end 14 fixed to an outer hub 16 and a distal end 18 defining an opening 20 forming a cutting port or window. Referring also to FIG. 2, an elongate tubular inner member 22 is rotatably received in outer tubular member 12. Tubular inner member 22 has a proximal end 24 fixed to an inner hub 26, and a distal end 28 having a cutting edge 30. Cutting edge 30 defines an aperture 31 communicating with a lumen 46 defined in tubular inner member 22. Inner hub 26 includes a tang 36 adapted to be driven by an electric motor (not shown) to rotate tubular inner member 22. Alternatively, tubular inner member 22 undergoes axial, reciprocating motion within outer member 12. When blade 10 is assembled, inner hub 26 is received in outer hub 16 and cutting edge 30 is positioned adjacent opening 20 of outer member 12.

Outer member 12 and inner member 22 are formed from electropolished stainless steel, e.g., hardened stainless steel such as 400 series stainless steel, or soft stainless steel such as 300 series stainless steel. The distal ends 18, 28 of outer and inner members 12, 22, respectively, are spaced close together, e.g., with a clearance within about 0.0001″ to 0.002″, preferably about 0.00075″ to 0.00175″, in order to provide optimum cutting action.

Referring to FIG. 3, a bearing surface coating 40 of polymer is formed on an outer surface 42 of inner member 22. The polymer is a lubricious polymer including those selected from the group consisting of fluoropolymers, polyimide, polyamide, polyoxymethylene (acetal), polyorganosiloxane (silicone), hydrogels, and polyamide-imides.

For example, in one application, a primer is first applied on outer surface 42 and then a coating including polytetrafluoroethylene (PTFE) is formed over the primer. In particular, the PTFE coating is a Teflon® coating sold under the name SilverStone SUPRA®, a product of E.I. du Pont de Nemours and Company, Wilmington, Del. The coating is provided using conventional processes for applying such polymer materials to metal surfaces, including the Impreglon II process, developed by Advanced Technology Incorporated (Minneapolis, Minn.)

Preferably, coating 40 runs substantially the full length of outer surface 42 of inner member 22 from distal end 28, including cutting edge 30, to proximal end 24, and over the full circumference of the inner member. However, it may be sufficient to apply coating 40 only at the distal ends of the members if there is sufficient clearance along the remainder of the members to prevent contact between the remainder of the members during use. It may be sufficient to apply coating 40 less than over the full circumference of the inner member, e.g., as stripes. In certain embodiments, it is preferable to mask proximal end 24 where inner hub 26 contacts tubular inner member 22. Masking this portion of tubular inner member 22 ensures a better mechanical connection between tubular inner member 22 and inner hub 26.

Coating 40 preferably has a thickness of about 0.0005″ to 0.0025″ such that the outer diameter of inner tubular member 22 is substantially the same as the inner diameter of outer tubular member 12, with the coating 40 engaging the inner surface of the outer tubular member during use to form a bearing surface. As shown in FIG. 4, coating 40 may be applied to an inner surface 44 of outer member 12 or to both outer surface 42 of inner member 22 and inner surface 44 of outer member 12.

In operation, inner member 22 is rotatably driven in outer member 12 (up to high speeds in the range of about 1,000 rpm to 10,000 rpm) such that cutting edge 30 engages body tissue via cutting port or window 20. Cut tissue is aspirated through lumen 46 via aperture 31.

In the absence of a surface coating 40, shedding (the removal of material from the facing surfaces 44, 42 of the outer and inner members 12, 22, respectively) may occur due to contact between outer and inner members 12, 22. This is particularly problematic when outer and inner members 12, 22 are formed from soft stainless steel, are closely spaced, and are subjected to high loads encountered when using cutting blade 10 to cut hard tissue such as bone and cartilage. The load on the blade tends to cause bending of the members, increasing the contact force between the members and thus increasing the shedding. The bearing characteristics of a polymer coating including PTFE are sufficient to reduce wear of surfaces 40, 42, when cutting blade 10 is subjected to high load, limiting shearing, galling and seizing of blade 10. Furthermore, use of a polymer as the coating material eliminates tarnishing of the coated surface.

Cutting blade 10 can be sterilized by conventional techniques including the use of ethylene oxide gas, without changing the color of coating 40. Opening 20 can have any desired configuration to cooperate with the configuration of the cutting edge or edges on the distal end of the inner tubular member so as to form trimmers, meniscus cutters, end cutters, side cutters, full radius cutters, synovial resectors, whiskers, open end cutters, arthroplasty burrs, slotted whiskers, tapered burrs, oval burrs, punch forceps, adenoidectomy cutters, and the like. The surgical cutting instrument of the present invention can have any desirable hub configuration to be utilized with any drive system or handpiece capable of rotating or reciprocating an elongate inner tubular member within an elongate outer tubular member to cut or otherwise engage body tissue at the distal end.

Other embodiments are within the scope of the following claims.

For example, instead of defining a lumen, inner member 22 can be solid. Inner member 22 can be in the form of an auger.

What is claimed is:

1. A surgical blade, comprising:
an elongate outer tubular member having an inner surface and defining a distal opening,
an elongate inner member having an outer surface, the elongate inner member being movably received within the outer tubular member, the elongate inner member including a distal cutter positionable adjacent the distal opening in the outer tubular member, and
a polymer coating disposed on a portion of the outer surface of the inner member, the inner surface of the outer tubular member, or both.

2. The surgical blade of claim 1 wherein the polymer coating is a lubricious polymer coating.

3. The surgical blade of claim 2 wherein the polymer coating comprises a polymer selected from the group consisting of fluoropolymers, polyimide, polyamide, polyoxymethylene, polyorganosiloxane, hydrogels, and polyamide-imide.

4. The surgical blade of claim 3 wherein the polymer coating comprises polytetrafluoroethylene.

5. The surgical blade of claim 1 wherein the coating is in a region of the distal opening of the outer tubular member, a region of the distal cutter of the inner member, or both.

6. The surgical blade of claim 1 wherein the coating is along substantially an entire length of the inner surface of the outer tubular member, the outer surface of the inner member, or both.

7. The surgical blade of claim 1 wherein the inner surface of the inner member defines a lumen.

8. The surgical blade of claim 1 wherein the inner member is received within the outer tubular member for rotation therein.

9. The surgical blade of claim 1 wherein the inner member is received within the outer tubular member for axial, reciprocating motion therein.

10. The surgical blade of claim 1 wherein a clearance between a distal region of the outer member and a distal region of the inner member prior to applying the coating is in a range of about 0.0001″ to 0.002″.

11. The surgical blade of claim 10 wherein the clearance is about 0.00075″ to 0.00175″.

12. The surgical blade of claim 1 wherein a thickness of the polymer coating is in a range of about 0.0005″ to 0.0025″.

13. The surgical blade of claim 1 wherein the outer tubular member and the inner member are formed from stainless steel.

14. A method of cutting tissue, comprising
providing a surgical blade including
an elongate outer tubular member having an inner surface and defining a distal opening,
an elongate inner member having an outer surface, the elongate inner member being movably received within the outer tubular member, the elongate inner member including a distal cutter positionable adjacent the distal opening in the outer tubular member to permit the cutter to engage body tissue through the distal opening, and
a polymer coating disposed on a portion of the outer surface of the inner tubular member, the inner surface of the outer tubular member, or both,
placing the cutting blade against the tissue, and
driving the inner member to cut the tissue.

15. The method of claim 14 wherein the polymer coating is a lubricious polymer coating.

16. The method of claim 15 wherein the polymer coating comprises a polymer selected from the group consisting of fluoropolymers, polyimide, polyamide, polyoxymethylene, polyorganosiloxane, hydrogels, and polyamide-imide.

17. The method of claim 15 wherein the polymer coating comprises polytetrafluoroethylene.

18. The method of claim 14 wherein the step of providing includes providing the inner and outer members with a clearance between a distal region of the outer member and a distal region of the inner member prior to applying the coating in a range of about 0.0001″ to 0.002″.

19. The method of claim 18 wherein the clearance is about 0.00075″ to 0.00175″.

20. The method of claim 14 wherein the step of providing includes providing the coating with a thickness in a range of about 0.0005″ to 0.0025″.

21. The method of claim 14 wherein the step of driving includes rotating the inner member within the outer member.

22. The method of claim 14 wherein the step of providing the surgical blade includes sterilizing the surgical blade.

23. A method of making a surgical blade, comprising:
providing an elongate outer tubular member having an inner surface and defining a distal opening,
providing an elongate inner member having an outer surface, the elongate inner member being movably received within the outer tubular member, the elongate inner member including a distal cutter positionable adjacent the distal opening in the outer tubular member to permit the cutter to engage body tissue through the distal opening, and
coating the inner surface of the outer tubular member, the outer surface of the inner tubular member, or both with a polymer.

24. The method of claim 23 wherein the polymer coating is a lubricious polymer coating.

25. The method of claim 24 wherein the polymer coating comprises a polymer selected from the group consisting of fluoropolymers, polyimide, polyamide, polyoxymethylene, polyorganosiloxane, hydrogels, and polyamide-imide.

26. The method of claim 24 wherein the polymer coating comprises polytetrafluoroethylene.
